(19) United States
(12) Patent Application Publication
(10) Pub. No.: US 2026/0114983 A1
(43) Pub. Date: Apr. 30, 2026

(54) BIODEGRADABLE PIEZOELECTRIC NERVE-GUIDANCE CONDUIT FOR PROMOTING REGENERATION OF SEVERED NERVE AND METHOD FOR MANUFACTURING THE SAME

(71) Applicant: UIF (University Industry Foundation), Yonsei University, Seoul (KR)

(72) Inventors: Sang Woo KIM, Seoul (KR); Sera JEON, Seoul (KR); Dabin KIM, Seoul (KR)

(73) Assignee: UIF (University Industry Foundation), Yonsei University, Seoul (KR)

(21) Appl. No.: 19/324,864

(22) Filed: Sep. 10, 2025

(30) Foreign Application Priority Data
Oct. 31, 2024 (KR) 10-2024-0152714

(51) Int. Cl. A61F 2/04; A61L 27/26; A61L 27/56; C08L 67/04

(52) U.S. Cl. CPC A61F 2/04; A61L 27/26; A61L 27/56; C08L 67/04; A61L 2430/32

(57) ABSTRACT

An aligned nanofiber-based nerve-guidance conduit is provided which acts as a structural guide for assisting regeneration of a damaged nerve, and which provides an environment suitable for nerve regeneration through a porous channel. The nerve-guidance conduit is made of a material having biodegradability so that no additional removal surgery is required even after nerve regeneration is completed. In addition, the nerve-guidance conduit is composed of a piezoelectric material that generates electricity during deformation. Thus, a scheme for promoting the regenerative ability of nerve cells through electrical stimulation generated when the guidance conduit is deformed is proposed.

### CROSS-REFERENCE TO RELATED APPLICATION

This application claims priority from Korean Patent Application No. 10-2024-0152714 filed on Oct. 31, 2024 in the Korean Intellectual Property Office, and all the benefits accruing therefrom under 35 U.S.C. 119, the contents of which in its entirety are herein incorporated by reference.

### BACKGROUND

### Field

The present disclosure relates to a biodegradable piezoelectric nerve-guidance conduit for promoting regeneration of a severed nerve and a method for manufacturing the same.

### Description of Related Art

Regeneration of severely damaged nerves, such as the severed nerve is made by extending the axons of the damaged nerve cells to fill the gap between the damaged areas. Damage to nerve tissue may result in decreased motor ability of muscles connected to the nerves, sensory paralysis, and loss of autonomic nervous system function. When the nerves are not connected to each other within a short period of time after the damage, it may be difficult to recover intact functions thereof. To solve this problem, a method for regenerating a damaged nerve includes a method for suturing both ends of the damaged nerve with each other or implanting one's own or another's nerve into the damaged area. However, when the area size of the damaged portion is large, it is difficult to apply this method, and there may be side effects such as permanent loss of function of the nerve donor site.

Recently, a nerve-guidance conduit is attracting attention as a new technology for nerve regeneration. The nerve-guidance conduit has a tube shape inserted into a severed nerve region, and serves to guide the growth direction of the nerve axon when the never axon is regenerated and to provide an environment suitable for nerve regeneration. There have been attempts to improve nerve regeneration ability by introducing a bio-derived material into the nerve-guidance conduit or implementing a physical structure to promote nerve regeneration through the nerve-guidance conduit. However, since the nerve-guidance conduit material generally does not have biodegradability, there is a hassle of the nerve-guidance conduit having to be removed through reoperation after nerve regeneration, and there is a problem in that nourishment helpful for nerve regeneration cannot be permeated through the nerve-guidance conduit.

Therefore, there is an urgent need to develop an advanced nerve-guidance conduit having a suitable physical form, biodegradability and permeability of the nourishment helpful for nerve regeneration, and functionality capable of constantly promoting the nerve regeneration after being inserted into the body. The nerve-guidance conduit should be naturally decomposed in the body, so that there is no need for additional surgery, and should be able to effectively deliver the nutrients and signaling molecules required in the nerve regeneration process. In addition, the physical structure of the nerve-guidance conduit should be able to effectively guide the growth direction of the axon, and should have biocompatibility to minimize the immune response.

### SUMMARY

A technical purpose of the present disclosure is to provide a nerve-guidance conduit capable of performing regenerative treatment based on electrical stimulation while having physical guidance, biodegradability, oxygen permeability, etc. for a severed nerve.

Purposes according to the present disclosure are not limited to the above-mentioned purpose. Other purposes and advantages according to the present disclosure that are not mentioned may be understood based on following descriptions, and may be more clearly understood based on embodiments according to the present disclosure. Further, it will be easily understood that the purposes and advantages according to the present disclosure may be realized using means shown in the claims or combinations thereof.

A first aspect of the present disclosure provides a biodegradable piezoelectric nerve-guidance conduit for promoting regeneration of a severed nerve, the biodegradable piezoelectric nerve-guidance conduit comprising: an outer conduit made of a biodegradable piezoelectric nanofiber film; and a plurality of inner conduits, each inner conduct being made of a biodegradable piezoelectric nanofiber film and having a diameter smaller than a diameter of the outer conduit, wherein the plurality of inner conduits are received in an inner space of the outer conduit, wherein a longitudinal direction of each of the inner conduits is parallel to an longitudinal direction of the outer conduit, wherein the inner conduits are arranged side by side in a cross-sectional direction of the outer conduit to form a multi-channel structure.

In accordance with some embodiments of the biodegradable piezoelectric nerve-guidance conduit, the biodegradable piezoelectric nanofiber film is made of a composite material including: a polymer, a low molecular weight compound, or a monomer exhibiting piezoelectric properties; and a polymer having hydrophilicity and flexibility.

In accordance with some embodiments of the biodegradable piezoelectric nerve-guidance conduit, the polymer exhibiting piezoelectric properties includes PLA (Polylactic Acid), PHB (Polyhydroxybutyrate), PHBV (Poly(hydroxybutyrate-co-hydroxyvalerate)), Silk (Fibroin), or PGA (Polyglycolic Acid), wherein the low molecule weight compound exhibiting piezoelectric properties includes diphenylalanine nanotube (FF-nanotube), wherein the monomer exhibiting piezoelectric properties includes glycine.

In accordance with some embodiments of the biodegradable piezoelectric nerve-guidance conduit, the polymer having hydrophilicity and flexibility includes polyethylene glycol (PEG), polyvinyl alcohol (PVA), poly(lactic-co-glycolic acid) (PLGA), polyethylene oxide (PEO), polydioxanone (PDO), or polycaprolactone (PCL).

In accordance with some embodiments of the biodegradable piezoelectric nerve-guidance conduit, the nerve-guidance conduit serves as a physical guide for nerve regeneration and at the same time, is capable of electrical stimulation using piezoelectric properties.

In accordance with some embodiments of the biodegradable piezoelectric nerve-guidance conduit, each of the outer conduit and the inner conduits is formed in a form of a cylindrical conduit, wherein each of the outer conduit and the inner conduits is able to transfer nutrients or oxygen for regeneration of the nerve to the nerve therethrough.

A second aspect of the present disclosure provides a method for manufacturing a biodegradable piezoelectric nerve-guidance conduit for promoting regeneration of a severed nerve, the method comprising: preparing a polymer composite material solution for manufacturing a biodegradable piezoelectric nanofiber film; electrospinning the polymer composite material solution to manufacture the biodegradable piezoelectric nanofiber film; shaping the biodegradable piezoelectric nanofiber film into a conduit shape to prepare an outer conduit and a plurality of inner conduits, wherein a diameter of the outer conduit is larger than a diameter of each of the plurality of inner conduits; and inserting the inner conduits into an inner space of the outer conduit such that a longitudinal direction of each of the inner conduits is parallel to an longitudinal direction of the outer conduit, wherein the inner conduits are arranged side by side in a cross-sectional direction of the outer conduit to form a multi-channel structure.

In accordance with some embodiments of the method for manufacturing the biodegradable piezoelectric nerve-guidance conduit, the biodegradable piezoelectric nanofiber film is made of a composite material including: a polymer, a low molecular weight compound, or a monomer exhibiting piezoelectric properties; and a polymer having hydrophilicity and flexibility.

In accordance with some embodiments of the method for manufacturing the biodegradable piezoelectric nerve-guidance conduit, the polymer exhibiting piezoelectric properties includes PLA (Polylactic Acid), PHB (Polyhydroxybutyrate), PHBV (Poly(hydroxybutyrate-co-hydroxyvalerate)), Silk (Fibroin), or PGA (Polyglycolic Acid), wherein the low molecule weight compound exhibiting piezoelectric properties includes diphenylalanine nanotube (FF-nanotube), wherein the monomer exhibiting piezoelectric properties includes glycine.

In accordance with some embodiments of the method for manufacturing the biodegradable piezoelectric nerve-guidance conduit, the polymer having hydrophilicity and flexibility includes polyethylene glycol (PEG), polyvinyl alcohol (PVA), poly(lactic-co-glycolic acid) (PLGA), polyethylene oxide (PEO), polydioxanone (PDO), or polycaprolactone (PCL).

In accordance with some embodiments of the method for manufacturing the biodegradable piezoelectric nerve-guidance conduit, the nerve-guidance conduit serves as a physical guide for nerve regeneration and at the same time, is capable of electrical stimulation using piezoelectric properties.

In accordance with some embodiments of the method for manufacturing the biodegradable piezoelectric nerve-guidance conduit, each of the outer conduit and the inner conduits is formed in a form of a cylindrical conduit, wherein each of the outer conduit and the inner conduits is able to transfer nutrients or oxygen for regeneration of the nerve to the nerve therethrough.

A third aspect of the present disclosure provides a biodegradable piezoelectric nerve-guidance conduit for promoting the regeneration of a severed nerve, the biodegradable piezoelectric nerve-guidance conduit being manufactured by the method for manufacturing the biodegradable piezoelectric nerve-guidance conduit for promoting regeneration of the severed nerve according to the second aspect, wherein a longitudinal direction of each of the inner conduits is parallel to an longitudinal direction of the outer conduit, wherein the inner conduits are arranged side by side in a cross-sectional direction of the outer conduit to form a multi-channel structure.

According to the present disclosure, the aligned nanofiber-based nerve-guidance conduit provides a structural guide for assisting the regeneration of a damaged nerve, and provides an environment suitable for nerve regeneration through the porous channel. The nerve-guidance conduit of the present disclosure is made of a material having biodegradability so that no additional removal surgery is required even after nerve regeneration is completed. In addition, since the nerve-guidance conduit is composed of a piezoelectric material that generates electricity during deformation, a method for promoting the regenerative ability of nerve cells through electrical stimulation generated when the guidance conduit is deformed is proposed.

According to the present disclosure, it is expected that aftereffects such as loss of function that may occur when the nerve is severed can be minimized, and the regeneration of damaged nerve tissue can be accelerated to recover the original function. The nerve-guidance conduit of the present disclosure guides the growth of nerve axons and provides physical and electrical stimulation suitable for nerve regeneration, thereby promoting nerve regeneration more effectively.

Effects of the present disclosure are not limited to the effects mentioned above, and other effects not mentioned will be clearly understood by those skilled in the art from the description as set forth below.

In addition to the above effects, specific effects of the present disclosure are described together while describing specific details for carrying out the present disclosure.

### BRIEF DESCRIPTION OF DRAWINGS

FIG. 1 illustrates a biodegradable piezoelectric nerve-guidance conduit for promoting regeneration of a severed nerve according to an embodiment of the present disclosure.

FIG. 2 illustrates an electric field generated along a longitudinal direction of a conduit made of piezoelectric nanofibers in a nerve-guidance conduit according to an embodiment of the present disclosure.

FIG. 3 is a flowchart illustrating a method for manufacturing a biodegradable piezoelectric nerve-guidance conduit for promoting regeneration of a severed nerve according to an embodiment of the present disclosure.

FIG. 4 is a diagram identifying that a voltage is generated due to piezoelectric characteristics when mechanical deformation occurs on the piezoelectric nerve-guidance conduit.

FIG. 5 shows a Toe-off phase behavior evaluation result in the evaluation experiment of the nerve regeneration.

FIG. 6 is a diagram illustrating an evaluation result of the nerve regeneration, and illustrates a result of analyzing the number and area size of axons based on Semi-thin section.

FIG. 7 illustrates a method for manufacturing a biodegradable piezoelectric nerve-guidance conduit.

### DETAILED DESCRIPTIONS

Advantages and features of the present disclosure, and a method of achieving the advantages and features will become apparent with reference to embodiments described later in detail together with the accompanying drawings. However, the present disclosure is not limited to the embodiments as disclosed under, but may be implemented in various different forms. Thus, these embodiments are set forth only to make the present disclosure complete, and to completely inform the scope of the present disclosure to those of ordinary skill in the technical field to which the present disclosure belongs, and the present disclosure is only defined by the scope of the claims.

For simplicity and clarity of illustration, elements in the drawings are not necessarily drawn to scale. The same reference numbers in different drawings represent the same or similar elements, and as such perform similar functionality. Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

A shape, a size, a ratio, an angle, a number, etc. disclosed in the drawings for illustrating embodiments of the present disclosure are illustrative, and the present disclosure is not limited thereto.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in the present disclosure, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of associated listed items. Expression such as “at least one of” when preceding a list of elements may modify the entire list of elements and may not modify the individual elements of the list. In interpretation of numerical values, an error or tolerance therein may occur even when there is no explicit description thereof.

When a certain embodiment may be implemented differently, a function or an operation specified in a specific block may occur in a different order from an order specified in a flowchart. For example, two blocks in succession may be actually performed substantially concurrently, or the two blocks may be performed in a reverse order depending on a function or operation involved.

When an embodiment may be implemented differently, functions or operations specified within a specific block may be performed in a different order from an order specified in a flowchart. For example, two consecutive blocks may actually be performed substantially simultaneously, or the blocks may be performed in a reverse order depending on related functions or operations.

The features of the various embodiments of the present disclosure may be partially or entirely combined with each other, and may be technically associated with each other or operate with each other. The embodiments may be implemented independently of each other and may be implemented together in an association relationship.

In interpreting a numerical value, the value is interpreted as including an error range unless there is no separate explicit description thereof. In the context of the present disclosure, the term “about” may mean about ±1%, about ±2%, about ±3%, about ±4%, about ±5%, about ±6%, about ±7%, about ±8%, about ±9%, or about ±10% of a value stated herein.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, “embodiments,” “examples,” “aspects, and the like should not be construed such that any aspect or design as described is superior to or advantageous over other aspects or designs.

The terms used in the description as set forth below have been selected as being general and universal in the related technical field. However, there may be other terms than the terms depending on the development and/or change of technology, convention, preference of technicians, etc. Therefore, the terms used in the description as set forth below should not be understood as limiting technical ideas, but should be understood as examples of the terms for illustrating embodiments.

In addition, it will also be understood that when a first element or layer is referred to as being present “on” a second element or layer, the first element may be disposed directly on the second element or may be disposed indirectly on the second element with a third element or layer being disposed between the first and second elements or layers. It will be understood that when a first element or layer is referred to as being “connected to”, or “coupled to” a second element or layer, the first element may be directly connected to or coupled to the second element or layer, or one or more intervening elements or layers may be present therebetween. In addition, it will also be understood that when an element or layer is referred to as being “between” two elements or layers, it may be the only element or layer between the two elements or layers, or one or more intervening elements or layers may also be present therebetween.

Further, as used herein, when a layer, film, area, plate, or the like is disposed “on” or “on a top” of another layer, film, area, plate, or the like, the former may directly contact the latter or still another layer, film, area, plate, or the like may be disposed between the former and the latter. As used herein, when a layer, film, area, plate, or the like is directly disposed “on” or “on a top” of another layer, film, area, plate, or the like, the former directly contacts the latter and still another layer, film, area, plate, or the like is not disposed between the former and the latter. Further, as used herein, when a layer, film, area, plate, or the like is disposed “below” or “under” another layer, film, area, plate, or the like, the former may directly contact the latter or still another layer, film, area, plate, or the like may be disposed between the former and the latter. As used herein, when a layer, film, area, plate, or the like is directly disposed “below” or “under” another layer, film, area, plate, or the like, the former directly contacts the latter and still another layer, film, area, plate, or the like is not disposed between the former and the latter.

In descriptions of temporal relationships, for example, temporal precedent relationships between two events such as “after”, “subsequent to”, “before”, etc., another event may occur therebetween unless “directly after”, “directly subsequent” or “directly before” is not indicated. When a certain embodiment may be implemented differently, a function or an operation specified in a specific block may occur in a different order from an order specified in a flowchart. For example, two blocks in succession may be actually performed substantially concurrently, or the two blocks may be performed in a reverse order depending on a function or operation involved.

In treating the nerve damage, rapid connection of the severed nerves to each other is a key factor not only in the regeneration of the nerves themselves, but also in preventing degeneration and sequelae of the connected muscle tissue thereto. The present disclosure proposes a nerve-guidance conduit which promotes nerve regeneration based on biodegradable piezoelectric nanofibers and is naturally decomposed in the body after the regeneration is completed.

The nerve-guidance conduit proposed in the present disclosure is manufactured by forming an aligned biodegradable piezoelectric nanofiber film into a cylindrical shape. In this case, the material of the conduit exhibits piezoelectric properties, and thus, electrical stimulation is generated when the conduit is physically deformed, and the regeneration of nerve cells along the aligned nanofibers may be promoted through the electrical stimulation. In addition, the porous structure of the nanofibers can smoothly supply nutrients and oxygen required for cell regeneration to damaged nerve regions, thereby supporting nerve regeneration. After the nerve regeneration is completed, the nerve-guidance conduit may be naturally decomposed in the body, or the decomposition rate may be accelerated through physical/chemical methods.

In this way, the present disclosure provides the advantage of promoting nerve regeneration and removing the conduit from the body without additional surgery, and will be described in detail below.

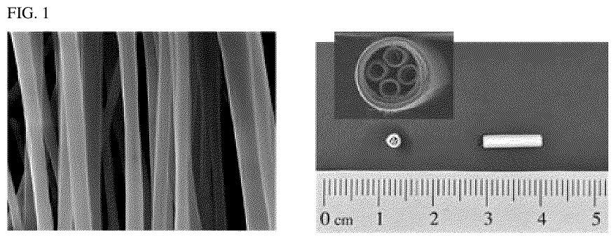

FIG. 1 illustrates a biodegradable piezoelectric nerve-guidance conduit for promoting regeneration of a severed nerve according to an embodiment of the present disclosure.

As shown in FIG. 1, the biodegradable piezoelectric nerve-guidance conduit for promoting regeneration of a severed nerve according to an embodiment of the present disclosure includes an outer conduit made of a biodegradable piezoelectric nanofiber film; and a plurality of inner conduits, each inner conduct being made of a biodegradable piezoelectric nanofiber film and having a diameter smaller than a diameter of the outer conduit, wherein the plurality of inner conduits are received in an inner space of the outer conduit, wherein a longitudinal direction of each of the inner conduits is parallel to an longitudinal direction of the outer conduit, wherein the inner conduits are arranged side by side in a cross-sectional direction of the outer conduit to form a multi-channel structure.

In accordance with some embodiments of the biodegradable piezoelectric nerve-guidance conduit, the biodegradable piezoelectric nanofiber film is made of a composite material including: a polymer, a low molecular weight compound, or a monomer exhibiting piezoelectric properties; and a polymer having hydrophilicity and flexibility.

In accordance with some embodiments of the biodegradable piezoelectric nerve-guidance conduit, the polymer exhibiting piezoelectric properties includes PLA (Polylactic Acid), PHB (Polyhydroxybutyrate), PHBV (Poly(hydroxybutyrate-co-hydroxyvalerate)), Silk (Fibroin), or PGA (Polyglycolic Acid), wherein the low molecule weight compound exhibiting piezoelectric properties includes diphenylalanine nanotube (FF-nanotube), wherein the monomer exhibiting piezoelectric properties includes glycine.

In accordance with some embodiments of the biodegradable piezoelectric nerve-guidance conduit, the polymer having hydrophilicity and flexibility includes polyethylene glycol (PEG), polyvinyl alcohol (PVA), poly(lactic-co-glycolic acid) (PLGA), polyethylene oxide (PEO), polydioxanone (PDO), or polycaprolactone (PCL).

The reason why the composite material is used is to compensate for the limitation of using only a material having piezoelectric properties. For example, PLA exhibiting piezoelectric properties exhibits slow biodegradation rate, hydrophobic properties, and brittle properties. In order to compensate for these shortcomings, the polymer (for example, PEG) having hydrophilicity and flexibility may be added thereto to form the composite material. Thus, the biodegradation rate thereof is improved, hydrophilicity is implemented, and ductility is improved, and thus the composite material is more suitable to be used as the material of the nerve-guidance conduit.

In accordance with some embodiments of the biodegradable piezoelectric nerve-guidance conduit, the nerve-guidance conduit serves as a physical guide for nerve regeneration and at the same time, is capable of electrical stimulation using piezoelectric properties.

In accordance with some embodiments of the biodegradable piezoelectric nerve-guidance conduit, each of the outer conduit and the inner conduits is formed in a form of a cylindrical conduit as shown in FIG. 1.

Each of the outer conduit and the inner conduits is able to transfer nutrients or oxygen for regeneration of the nerve to the nerve therethrough.

In addition, the nerve-guidance conduit of the present disclosure serves as a physical guide for nerve regeneration, and at the same time, electrical stimulation by piezoelectric properties is generated.

FIG. 1 shows aligned nanofibers and the appearance after forming the aligned nanofibers into a cylindrical shape. Nanofibers having a diameter of about 1 μm are highly aligned. The nerve-guidance conduit composed of such nanofibers has a multi-channel structure including four relatively small inner cylinders (for example, the number of the inner cylinders may be 4. However, the present disclosure is not limited thereto, and the number of the plurality of inner cylinders may be variable) received inside an outer cylinder having a large inner diameter. This multi-channel structure is designed to effectively guide the growth of axons in the process of nerve regeneration. In addition, the conduit of the present disclosure can utilize a piezoelectric phenomenon in which when a physical force is applied or ultrasonic waves are applied to the nanofibers, the nanofibers are deformed and thus an internal polarization is changed, thereby generating electricity in accordance with the application period of the physical stimulation, and the electrical stimulation can promote the regeneration of nerve cells.

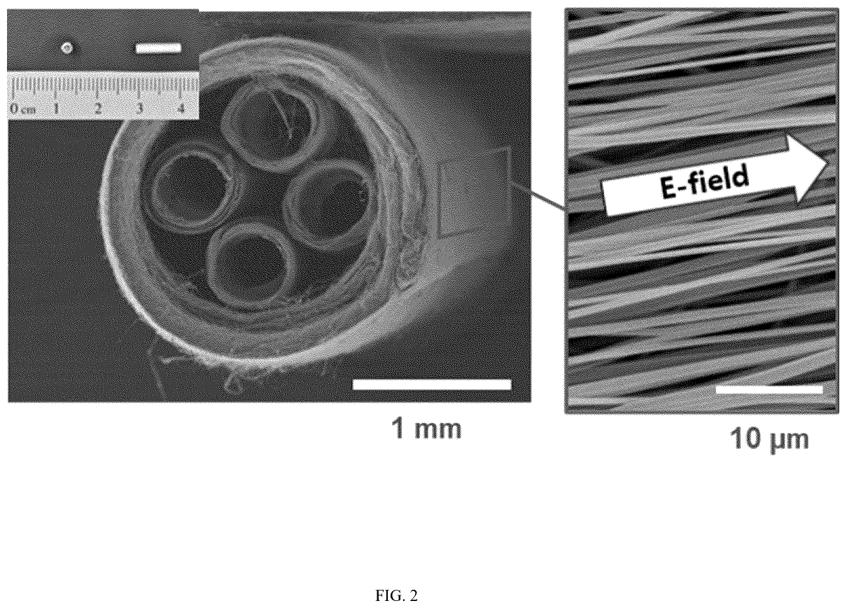

FIG. 2 illustrates an electric field generated along a longitudinal direction of a conduit made of piezoelectric nanofibers in a nerve-guidance conduit according to an embodiment of the present disclosure.

As shown in FIGS. 1 and 2, the plurality of inner conduits are received in an inner space of the outer conduit, wherein a longitudinal direction of each of the inner conduits is parallel to an longitudinal direction of the outer conduit, wherein the inner conduits are arranged side by side in a cross-sectional direction of the outer conduit to form a multi-channel structure. Such a nerve-guidance conduit has a technically important meaning in that it not only serves as a physical guide for nerve regeneration, but also applies electrical stimulation to accelerate the nerve cell regeneration. This can simultaneously provide two therapeutic effects: promoting physical regeneration and promoting electrical regeneration. Thus, the nerve-guidance conduit according to an embodiment of the present disclosure is evaluated as an innovative approach in the treatment of nerve damage. In addition, according to the present disclosure, the nerve-guidance conduit is fabricated based on the biodegradable material. After the nerve regeneration is completed, the nerve-guidance conduit can be naturally decomposed in the body without an additional removal surgery, thereby solving the problem that the nerve-guidance conduit remains in the body and significantly reducing the economic and physical burden of a patient due to additional surgery.

The biodegradable piezoelectric nerve-guidance conduit for promoting regeneration of a severed nerve according to an embodiment of the present disclosure has been described above. Hereinafter, a method for manufacturing a biodegradable piezoelectric nerve-guidance conduit for promoting regeneration of a severed nerve according to an embodiment of the present disclosure will be described. The contents duplicate with those as described above will be briefly described or the descriptions thereof will be omitted.

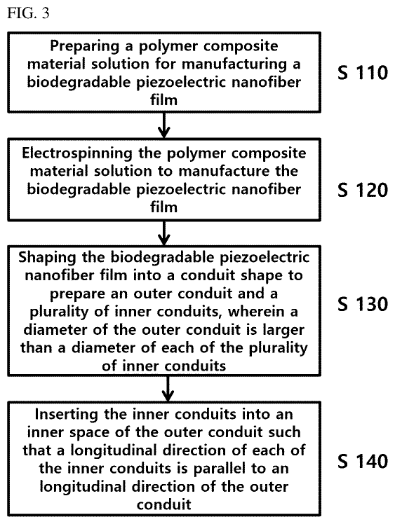

FIG. 3 is a flowchart illustrating a method for manufacturing a biodegradable piezoelectric nerve-guidance conduit for promoting regeneration of a severed nerve according to an embodiment of the present disclosure.

Referring to FIG. 3, the method for manufacturing the biodegradable piezoelectric nerve-guidance conduit for promoting regeneration of a severed nerve according to an embodiment of the present disclosure includes preparing a polymer composite material solution for manufacturing a biodegradable piezoelectric nanofiber film in S 110; electrospinning the polymer composite material solution to manufacture the biodegradable piezoelectric nanofiber film in S 120; shaping the biodegradable piezoelectric nanofiber film into a conduit shape to prepare an outer conduit and a plurality of inner conduits, wherein a diameter of the outer conduit is larger than a diameter of each of the plurality of inner conduits in S 130; and inserting the inner conduits into an inner space of the outer conduit such that a longitudinal direction of each of the inner conduits is parallel to an longitudinal direction of the outer conduit, wherein the inner conduits are arranged side by side in a cross-sectional direction of the outer conduit to form a multi-channel structure in S 140.

In step S110, the polymer composite material solution for manufacturing the biodegradable piezoelectric nanofiber film is prepared. The polymer composite material solution is prepared by mixing a polymer, a low molecular weight compound, or a monomer exhibiting piezoelectric properties with a polymer having hydrophilicity and flexibility in an organic solvent, and stirring and dissolving the mixture.

In accordance with some embodiments of the method for manufacturing the biodegradable piezoelectric nerve-guidance conduit, the polymer exhibiting piezoelectric properties includes PLA (Polylactic Acid), PHB (Polyhydroxybutyrate), PHBV (Poly (hydroxybutyrate-co-hydroxyvalerate)), Silk (Fibroin), or PGA (Polyglycolic Acid), wherein the low molecule weight compound exhibiting piezoelectric properties includes diphenylalanine nanotube (FF-nanotube), wherein the monomer exhibiting piezoelectric properties includes glycine.

In accordance with some embodiments of the method for manufacturing the biodegradable piezoelectric nerve-guidance conduit, the polymer having hydrophilicity and flexibility includes polyethylene glycol (PEG), polyvinyl alcohol (PVA), poly (lactic-co-glycolic acid) (PLGA), polyethylene oxide (PEO), polydioxanone (PDO), or polycaprolactone (PCL).

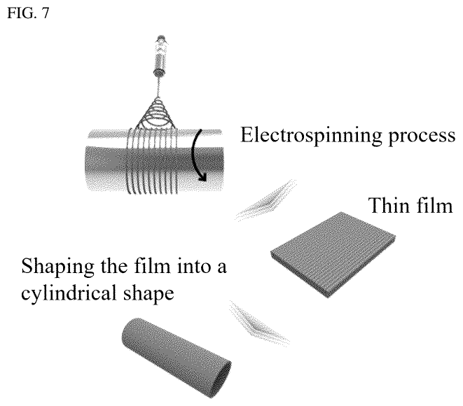

In step S120, the biodegradable piezoelectric nanofiber film is manufactured by electrospinning the polymer composite material solution. The polymer composite material solution is synthesized into nanofibers in an electrospinning manner. Then, the nanofibers are formed into the biodegradable piezoelectric nanofiber film (an electrospun mat) in which the nanofibers are stacked. In this case, the biodegradable piezoelectric nanofiber film (the electrospun mat) refers to a material such as cloth. As shown in FIG. 7, the biodegradable piezoelectric nanofiber film may be formed in a form of a thin film in an electrospinning process.

In step S130, the biodegradable piezoelectric nanofiber film is shaped into the form of the conduit to prepare an outer conduit having a large diameter and a plurality of inner conduits having a small diameter, respectively. The nanofiber film may be cut into appropriate sizes and may be wound on a rod while applying heat thereto to shape the biodegradable piezoelectric nanofiber film into the form of the conduit. In this case, by applying the heat to the film, not only the shape of the conduit into which the nanofiber film is shaped may be fixed, but also the crystallinity of the polymer may be increased, thereby improving piezoelectric performance. When the heat is applied, the appropriate temperature range may be in a range of 80° C. exclusive to 140° C. exclusive. The crystallization of the polymer is not increased when the temperature is equal to or lower than 80° C. On the contrary, there is a possibility of melting of PLLA when the temperature is equal to or higher than 140° C. As shown in FIG. 7, the film may be shaped into the form of the conduit.

In step S140, the inner conduits may be inserted into an inner space of the outer conduit such that a longitudinal direction of each of the inner conduits is parallel to an longitudinal direction of the outer conduit, wherein the inner conduits are arranged side by side in a cross-sectional direction of the outer conduit to form a multi-channel structure. For example, as shown in FIG. 1, four inner conduits may be received in the inner space of one outer conduit having a large diameter. For example, the number of the inner cylinders may be 4. However, the present disclosure is not limited thereto, and the number of the plurality of inner cylinders may be variable.

Hereinafter, more specific Examples and experimental Examples will be described. However, the following Examples are merely some embodiments of the present disclosure, and the scope of the present disclosure is not limited to the following Examples.

### EXAMPLE 1

In Example 1, PLA was used as the polymer material exhibiting piezoelectric properties, and PEG was used as the polymer material having hydrophilicity and flexibility. That is, a poly-lactide (PLA)/polyethylene glycol (PEG) composite material was used as the nerve-guidance conduit material.

PLLA (Poly-L-lactic Acid) and PEG (Polyethylene Glycol) were mixed with each other in an organic solvent (dichloromethane:DCM) at a 1:1 ratio, and then the mixture was stirred at 120° C. to dissolve the mixture, thereby preparing the polymer composite material solution. In this case, PLLA and PEG may be replaced with different materials, and accordingly, the used organic solvent may also be changed to a solvent suitable for the replacing materials.

The prepared polymer composite material solution was synthesized into nanofibers using electrospinning, and the nanofibers was formed into the nanofiber film in which the nanofibers are stacked. Such a nanofiber film has a texture like a cloth. The film was cut into an appropriate size. While the heat was applied thereto, the heated film was wound on a thin metal rod such that the film was shaped into the form of the nerve-guidance conduit. At this time, the structural stability of the nerve-guidance conduit can be secured by fixing the shape of the conduit into which the nanofiber had been shaped through the process of applying the heat to the film, and the crystallinity of the polymer can be increased in the heat applying process, thereby improving the piezoelectric performance of the nerve-guidance conduit.

The finally produced nerve-guidance conduit has a multi-channel structure in which the four nerve-guidance inner conduits with a small diameter were inserted into the single nerve-guidance outer conduit with a large diameter.

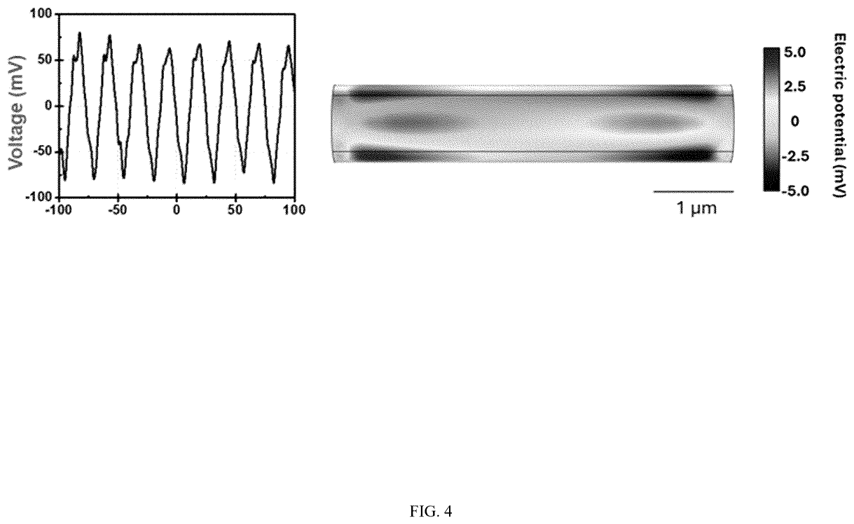

FIG. 4 is a diagram identifying that a voltage is generated due to the piezoelectric characteristics when mechanical deformation occurs on the piezoelectric nerve-guidance conduit. In FIG. 4, the fact that the voltage is generated due to the piezoelectric characteristics when mechanical deformation occurs on the piezoelectric nerve-guidance conduit was identified by applying the ultrasonic waves thereto.

### EXAMPLE 2

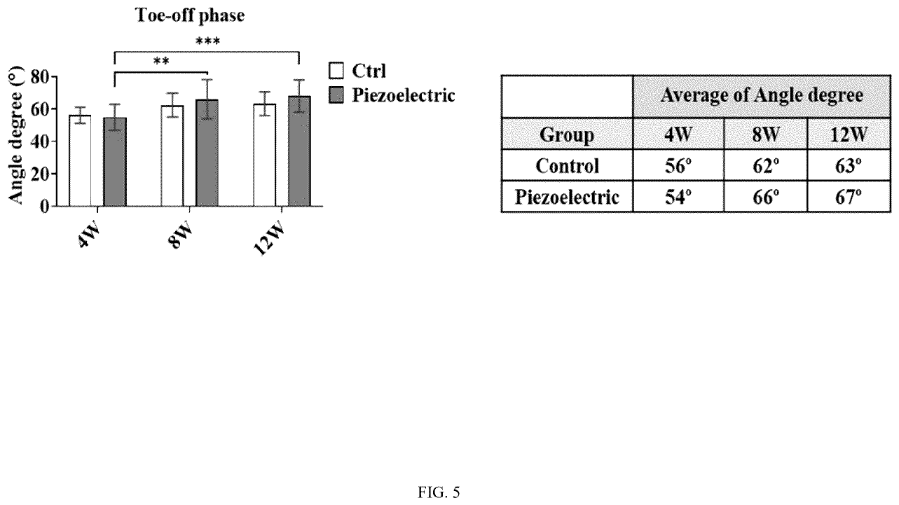

In order to verify the technical validity of the nerve-guidance conduit of the present disclosure, an animal experiment was conducted on the regeneration effect of the severed nerve. FIG. 5 shows a Toe-off phase behavior evaluation result in the evaluation of the neural regeneration in the animal experiment. In the experiment, the sciatic nerve of an 8-week-old rat was cut to a length of 8 mm, and then the biodegradable piezoelectric nanofiber-based nerve-guidance conduit was inserted into the severed site. The severed nerve area was fixed into the nerve-guidance conduit through suturing, so that nerve regeneration was performed accurately in the nerve-guidance conduit

Thereafter, ultrasonic waves were applied to the conduit every other day to generate electrical output therefrom while the piezoelectric nanofibers in the body were deformed. The electrical output generated therefrom under the ultrasonic stimulation played an important role in promoting nerve regeneration by providing electrical stimulation to the damaged nerve area. Thus, the nerve regeneration was accelerated, and the recovery process of the severed nerve tissue was monitored.

In order to verify the effect and effectiveness of the nerve-guidance conduit of the present disclosure, a comparative experiment was conducted by setting the autologous nerve graft as a control group. In the control group, the nerve was cut to the same length, and autologous tissues were sutured and connected to the severed nerve. To compare the neuroregenerative effects of the neural-guidance conduit group and the autologous nerve graft control group, the experiment was conducted based on behavioral assessment by gait analysis over weeks 4, 8, and 12 and pathological assessment via cross-sectional analysis of the sciatic nerve regenerated at 12 weeks. According to the result of the Toe off phase behavior analysis of FIG. 5, the neural regeneration exhibited similar values in both the neural guidance conduit group and the autologous nerve graft control group through gait evaluation at week 4, week 8, and week 12. This indicates that the progress of nerve regeneration was performed stably in the both groups.

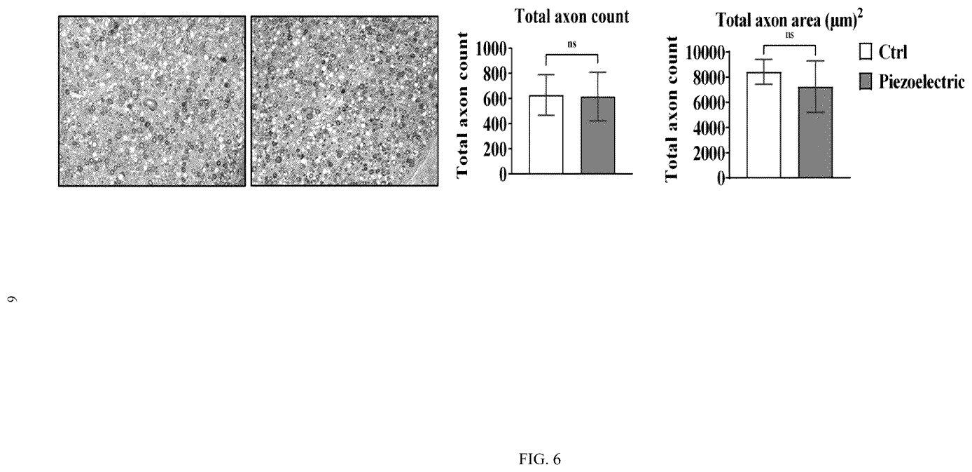

In addition, FIG. 6 is a diagram showing the evaluation of the neural regeneration, and shows the results of analyzing the number and the area size of axons based on Semi-thin section. As a result of evaluating the number and area of axons based on the cross-sectional image of the Toluidine blue-stained sciatic nerve in FIG. 6, it was identified that the difference between the two groups was not statistically significant. Thus, it was experimentally verified that the piezoelectric nanofiber-based nerve-guidance conduit of the present disclosure can accelerate nerve regeneration to the extent comparable to the autologous nerve graft control group. Accordingly, it was identified that the nerve-guidance conduit of the present disclosure can be an excellent alternative for effectively promoting nerve regeneration without additional surgery compared to the autologous nerve graft.

Although the embodiments of the present disclosure have been described above with reference to the accompanying drawings, the present disclosure may not be limited to the embodiments and may be implemented in various different forms. Those of ordinary skill in the technical field to which the present disclosure belongs will be able to appreciate that the present disclosure may be implemented in other specific forms without changing the technical idea or essential features of the present disclosure. Therefore, it should be understood that the embodiments as described above are not restrictive but illustrative in all respects.

What is claimed is:

1. A biodegradable piezoelectric nerve-guidance conduit for promoting regeneration of a severed nerve, the biodegradable piezoelectric nerve-guidance conduit comprising:
an outer conduit made of a biodegradable piezoelectric nanofiber film; and
a plurality of inner conduits, each inner conduct being made of a biodegradable piezoelectric nanofiber film and having a diameter smaller than a diameter of the outer conduit,
wherein the plurality of inner conduits are received in an inner space of the outer conduit,
wherein a longitudinal direction of each of the inner conduits is parallel to an longitudinal direction of the outer conduit,
wherein the inner conduits are arranged side by side in a cross-sectional direction of the outer conduit to form a multi-channel structure.

2. The biodegradable piezoelectric nerve-guidance conduit of claim 1, wherein the biodegradable piezoelectric nanofiber film is made of a composite material including:
a polymer, a low molecular weight compound, or a monomer exhibiting piezoelectric properties; and
a polymer having hydrophilicity and flexibility.

3. The biodegradable piezoelectric nerve-guidance conduit of claim 2, wherein the polymer exhibiting piezoelectric properties includes PLA (Polylactic Acid), PHB (Polyhydroxybutyrate), PHBV (Poly (hydroxybutyrate-co-hydroxyvalerate)), Silk (Fibroin), or PGA (Polyglycolic Acid),
wherein the low molecule weight compound exhibiting piezoelectric properties includes diphenylalanine nanotube (FF-nanotube),
wherein the monomer exhibiting piezoelectric properties includes glycine.

4. The biodegradable piezoelectric nerve-guidance conduit of claim 2, wherein the polymer having hydrophilicity and flexibility includes polyethylene glycol (PEG), polyvinyl alcohol (PVA), poly(lactic-co-glycolic acid) (PLGA), polyethylene oxide (PEO), polydioxanone (PDO), or polycaprolactone (PCL).

5. The biodegradable piezoelectric nerve-guidance conduit of claim 1, wherein the nerve-guidance conduit serves as a physical guide for nerve regeneration and at the same time, is capable of electrical stimulation using piezoelectric properties.

6. The biodegradable piezoelectric nerve-guidance conduit of claim 1, wherein each of the outer conduit and the inner conduits is formed in a form of a cylindrical conduit,
wherein each of the outer conduit and the inner conduits is able to transfer nutrients or oxygen for regeneration of the nerve to the nerve therethrough.

7. A method for manufacturing a biodegradable piezoelectric nerve-guidance conduit for promoting regeneration of a severed nerve, the method comprising:
preparing a polymer composite material solution for manufacturing a biodegradable piezoelectric nanofiber film;
electrospinning the polymer composite material solution to manufacture the biodegradable piezoelectric nanofiber film;
shaping the biodegradable piezoelectric nanofiber film into a conduit shape to prepare an outer conduit and a plurality of inner conduits, wherein a diameter of the outer conduit is larger than a diameter of each of the plurality of inner conduits; and
inserting the inner conduits into an inner space of the outer conduit such that a longitudinal direction of each of the inner conduits is parallel to an longitudinal direction of the outer conduit,
wherein the inner conduits are arranged side by side in a cross-sectional direction of the outer conduit to form a multi-channel structure.

8. The method for manufacturing the biodegradable piezoelectric nerve-guidance conduit of claim 7, wherein the biodegradable piezoelectric nanofiber film is made of a composite material including:
a polymer, a low molecular weight compound, or a monomer exhibiting piezoelectric properties; and
a polymer having hydrophilicity and flexibility.

9. The method for manufacturing the biodegradable piezoelectric nerve-guidance conduit of claim 8, wherein the polymer exhibiting piezoelectric properties includes PLA (Polylactic Acid), PHB (Polyhydroxybutyrate), PHBV (Poly (hydroxybutyrate-co-hydroxyvalerate)), Silk (Fibroin), or PGA (Polyglycolic Acid),
wherein the low molecule weight compound exhibiting piezoelectric properties includes diphenylalanine nanotube (FF-nanotube),
wherein the monomer exhibiting piezoelectric properties includes glycine.

10. The method for manufacturing the biodegradable piezoelectric nerve-guidance conduit of claim 8, wherein the polymer having hydrophilicity and flexibility includes polyethylene glycol (PEG), polyvinyl alcohol (PVA), poly(lactic-co-glycolic acid) (PLGA), polyethylene oxide (PEO), polydioxanone (PDO), or polycaprolactone (PCL).

11. The method for manufacturing the biodegradable piezoelectric nerve-guidance conduit of claim 7, wherein the nerve-guidance conduit serves as a physical guide for nerve regeneration and at the same time, is capable of electrical stimulation using piezoelectric properties.

12. The method for manufacturing the biodegradable piezoelectric nerve-guidance conduit of claim 7, wherein each of the outer conduit and the inner conduits is formed in a form of a cylindrical conduit,
wherein each of the outer conduit and the inner conduits is able to transfer nutrients or oxygen for regeneration of the nerve to the nerve therethrough.
